(19) United States
(12) Patent Application Publication
(10) Pub. No.: US 2017/0333247 A1
(43) Pub. Date: Nov. 23, 2017

(54) COOLING PACK FOR HEAD PROTECTOR

(71) Applicant: Changui Industry Co., Ltd., Daejeon (KR)

(72) Inventors: Seong Nam JEON, Daejeon (KR); Soon Hak CHO, Seoul (KR)

(73) Assignee: Changui Industry Co., Ltd., Daejeon (KR)

(21) Appl. No.: 15/355,720

(22) Filed: Nov. 18, 2016

(30) Foreign Application Priority Data
May 19, 2016 (KR) 10-2016-0061225

(51) Int. Cl. A61F 7/02; A42B 1/00; A42B 3/12; A42B 3/28; A61F 7/00

(52) U.S. Cl. CPC A61F 7/02; A42B 3/121; A42B 3/285; A42B 1/008; A61F 2007/0008; A61F 2007/0226; A61F 2007/0228; A61F 2007/0266; A61F 2007/0214

(57) ABSTRACT

Disclosed is a cooling pack for a head including an outer cover fixed at an inside of the head protector. The outer cover adheres to a top of a human body, a refrigerant is embedded in the outer cover, and a plurality of fixers are coupled to the outer cover. The fixers are attachable to or detachable from the head protector, and the refrigerant is formed of a polyester polymer powder.

### CROSS-REFERENCE TO RELATED APPLICATION

This application claims the priority benefit of Korean Patent Application No. 10-2016-0061225, filed on May 19, 2016 in the Korean Intellectual Property Office, the disclosure of which is incorporated herein by reference.

### BACKGROUND OF THE INVENTION

### 1. Field of the Invention

The present invention relates to a cooling pack for a head protector, more particularly, to a cooling pack for a head protector in which a cooling pack in a water-absorbed state is coupled to the head protector to adhere to a top of a head, thereby preventing temperature rise of a scalp by evaporation of absorbed water when wearing the head protector to provide comfortable fit to a user.

### 2. Description of the Related Art

In general, a head protector is classified into a safety helmet, a baseball cap, an alpine hat, and a helmet for a vehicle or a motorcycle.

Such a head protector has no structure or device for cooling heat generated from a scalp such that it is difficult to wear the head protector for hours in the summer.

Particularly, the safety helmet should be worn for hours to safely protect a head from impact or fall in various industrial settings.

To this end, the safety helmet is manufactured using a thin and strong material such as synthetic resins or carbon resins to secure activity of a user wearing the safety helmet. However, due to shortage of ventilation in the safety helmet, temperature in the safety helmet is higher than in ambient air.

Accordingly, when wearing the safety helmet for hours, the user may suffer from headache. Thus, the user cannot focus on working. Finally, in severe cases, the user discontinues work. Thereby, productivity is decreased due to decreased work efficiency and economic loss due to construction delay is generated.

In particular, when the safety helmet is worn in the case of high ambient temperature in hot summer, sweat comes out of an inside of the helmet and rolls down the user's face, blocking the user's view, thereby interfering with operation and work. If the user does not wear the safety helmet due to the above problem, a worse accident may occur.

### SUMMARY OF THE INVENTION

Therefore, the present invention has been made in view of the above problems, and it is an object of the present invention to provide a cooling pack for a head protector preventing temperature rise of a scalp when wearing the head protector, thereby providing comfortable fit to a user.

It is another object of the present invention to provide a cooling pack for a head protector functioning as a buffer material capable of protecting the head from external impact using increase in volume when absorbing water.

In accordance with the present invention, the above and other objects can be accomplished by the provision of a cooling pack for a head protector including an outer cover fixed at an inside of the head protector, the outer cover adhering to a top of a human body, a refrigerant embedded in the outer cover, and a plurality of fixers coupled to the outer cover, the fixers being attachable to or detachable from the head protector, wherein the refrigerant is formed of a polyester polymer powder.

The outer cover may be formed of nylon or a mixture of nylon and polyurethane in a ratio 9:1.

The fixers may include at least one selected from Velcro, a button, a zipper, a press button, a strap, a rubber band, or a cable tie.

The cooling pack may have at least one shape selected from a disc shape, a ring shape, a tetragonal shape, a triangular shape, a hexagonal shape, an octagonal shape, and a dome shape.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a cooling pack for a head protector according to an embodiment of the present invention;

FIG. 2 is a cross-sectional view illustrating a safety helmet applying the cooling pack for the head protector according to the illustrated embodiment of the present invention; and

FIG. 3 is an exemplary view illustrating the cooling pack in a fixed state to the head protector of FIG. 2.

### DETAILED DESCRIPTION OF THE PREFERRED EMBODIMENTS

Hereinafter it will be described together with the accompanying drawings and with respect to a cooling pack for a head protector according to the present invention.

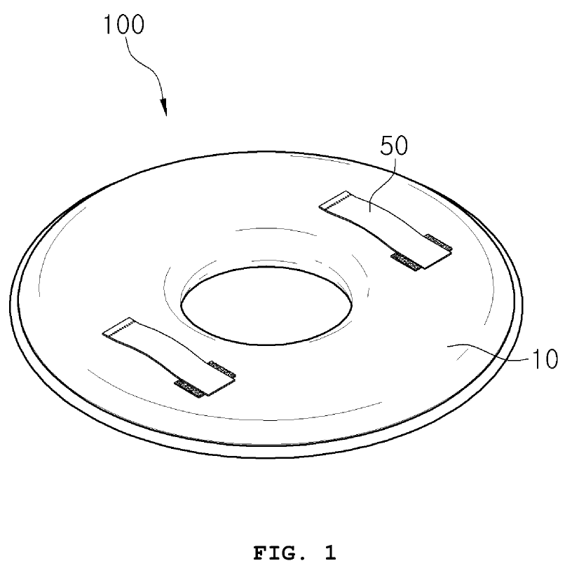

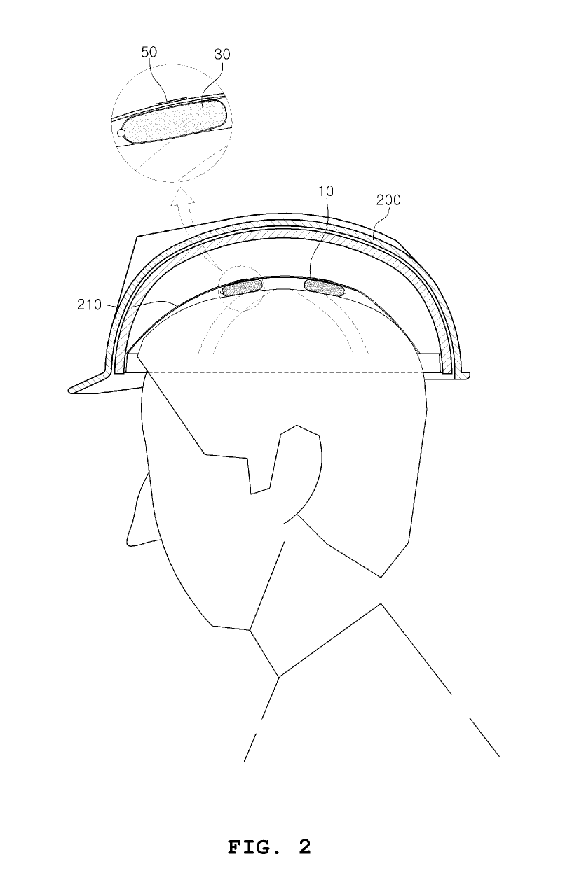

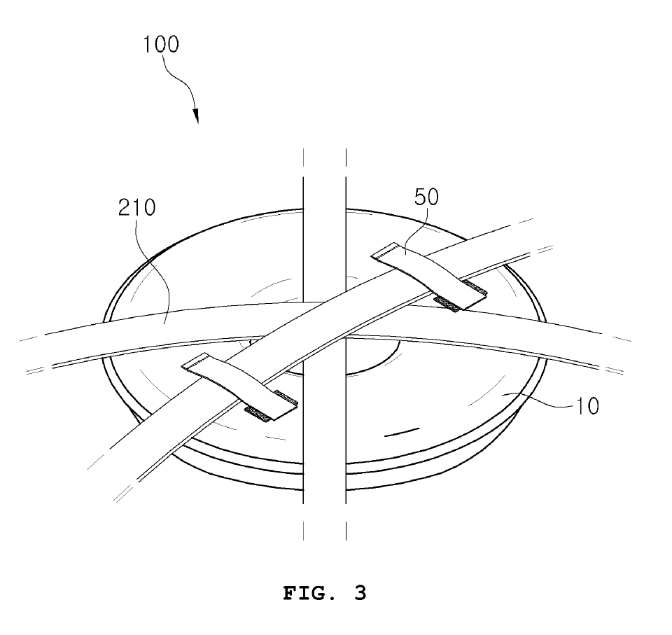

FIG. 1 is a perspective view of a cooling pack for a head protector according to an embodiment of the present invention. FIG. 2 is a cross-sectional view illustrating a safety helmet applying the cooling pack for the head protector according to the illustrated embodiment of the present invention. FIG. 3 is an exemplary view illustrating the cooling pack in a fixed state to the head protector of FIG. 2.

As illustrated in FIGS. 1 to 3, according to the present invention, the cooling pack 100 in a water-absorbed state is coupled to the head protector to adhere to a top of a human body. When wearing the head protector with the cooling pack 100, temperature rise of a scalp may be prevented by evaporation of absorbed water, thereby providing comfortable fit to a user.

The cooling pack 100 for the head protector mainly includes three parts, which are an outer cover 10, a refrigerant 30, and a fixer.

The outer cover 10 is fixed at an inside of the head protector to adhere to a top of the human body. The outer cover 10 may have at least one shape selected from a disc shape, a ring shape, a tetragonal shape, a triangular shape, a hexagonal shape, an octagonal shape, and a dome shape.

In the illustrated embodiment of the present invention, the outer cover 10 has a “ring” shape. Thus, manufacturing costs may be decreased and evaporation of the refrigerant 30 embedded in the outer cover 10 may be generated in three dimensions.

In addition, the outer cover 10 of the illustrated embodiment of the present invention may be formed of nylon having high air and water permeability, or a mixture of nylon and polyurethane in a ratio of 9:1.

Herein, when the mixture of nylon and polyurethane is used, the outer cover 10 may have elasticity and tensile strength, which are characteristics of polyurethane.

Furthermore, the outer cover 10 may be formed of polyester, ramie fabric, or cotton, and may be formed of a mixture having at least two of the above elements.

The refrigerant 30 is embedded in the outer cover 10. The refrigerant 30 is formed of a polyester polymer powder.

The polyester polymer is non-toxic and is capable of absorbing water until a volume thereof increases by about 30 to 50 times the original volume.

In addition, when the refrigerant 30 absorbs water, the volume thereof increases such that there is an additional effect, namely, the cooling pack 100 functions as a buffer material when the cooling pack 100 is applied to a safety helmet 200, thereby protecting the head from external impact.

The fixer is coupled to the outer cover 10 of the cooling pack 100 while being attachable to or detachable from the inside of the head protector.

A plurality of fixers may be provided. The fixer may include at least one selected from Velcro 50, a button, a zipper, a press button, a strap, a rubber band, or a cable tie.

As illustrated in FIGS. 2 and 3, the fixer may include the Velcro 50 in the illustrated embodiment of the present invention.

Herein, when the cooling pack 100 is applied to a safety helmet, after the cooling pack 100 for the head protector is immersed in water to absorb water, the cooling pack 100 in a water-absorbed state may be configured to be attachable to or detachable from a head support strap 210 of the safety helmet by coupling male/female of the Velcro 50.

Meanwhile, the head protector may include a baseball cap, an alpine hat, and a helmet for a vehicle or a motorcycle, instead of the safety helmet 200.

Herein, when the cooling pack 100 is mounted at a baseball cap or a helmet, the fixer may be coupled to a friction pad (not shown). Alternatively, a concavo-convex part may be formed, thereby preventing the cooling pack 100 from moving in the baseball cap or the helmet using friction force of the concavo-convex part.

Thus, the present invention has been described in the embodiment shown in the drawings by reference, which is merely illustrative and, those skilled in the art understand that various modifications and equivalent other embodiments are possible will.

As is apparent from the above description, according to the cooling pack for the head protector, it may be easy to apply the pre-existing head protector.

In addition, the cooling pack in a water-absorbed state is coupled to the head protector, such that temperature rise of the scalp, when wearing the head protector, may be prevented due to evaporation of absorbed water, thereby providing comfortable fit to a user.

In addition, the cooling pack for the head protector may function as a buffer material capable of protecting the head from external impact using increase in volume when the cooling pack absorbs water.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A cooling pack for a head protector comprising:
an outer cover fixed at an inside of the head protector, the outer cover adhering to a top of a human body;
a refrigerant embedded in the outer cover; and
a plurality of fixers coupled to the outer cover, the fixers being attachable to or detachable from the head protector,
wherein the refrigerant is formed of a polyester polymer powder.

2. The cooling pack for the head protector according to claim 1, wherein the outer cover is formed of nylon or a mixture of nylon and polyurethane in a ratio 9:1.

3. The cooling pack for the head protector according to claim 1, wherein the fixers include at least one selected from Velcro, a button, a zipper, a press button, a strap, a rubber band, or a cable tie.

4. The cooling pack for the head protector according to claim 1, wherein the cooling pack has at least one shape selected from a disc shape, a ring shape, a tetragonal shape, a triangular shape, a hexagonal shape, an octagonal shape, and a dome shape.
